(19) United States
(12) Patent Application Publication
(10) Pub. No.: US 2015/0182202 A1
(43) Pub. Date: Jul. 2, 2015

(54) EXCREMENT COLLECTOR

(71) Applicant: W.H.P.M. BIORESEARCH AND TECHNOLOGY CO., LTD., Beijing (CN)

(72) Inventors: John Wan, Beijing (CN); Chunhua Yuan, Beijing (CN); Qinghai Xia, Beijing (CN)

(73) Assignee: W.H.P.M. BIORESEARCH AND TECHNOLOGY CO., LTD., Beijing (CN)

(21) Appl. No.: 14/380,188

(22) PCT Filed: Feb. 21, 2013
(86) PCT No.: PCT/CN2013/071737

§ 371 Date: Aug. 21, 2014

(30) Foreign Application Priority Data
Feb. 24, 2012 (CN) 201210045214.1

(51) Int. Cl. A61B 10/00

(52) U.S. Cl. CPC A61B 10/0038

(57) ABSTRACT

An excrement collector comprises an upper cover (100), a collecting tube (300), a bottom cover (400) and a filtering tube (200), wherein the filtering tube (200) is fixed on the upper side inside the tube cavity of the collecting tube (300); a sampling end (103) is formed on the lower part of the upper cover (100) and passes through a through hole (203) at the bottom of the filtering tube (200); the upper cover (100) is detachably integrated with the upper part of the collecting tube (300); the bottom cover (400) is detachably integrated with the lower part of the collecting tube (300); and liquid is provided in the cavity between the collecting tube (300) and the filtering tube (200).

### CROSS REFERENCE TO RELATED APPLICATIONS

This application claims the priority of the Chinese patent application 201210045214.1 submitted on Feb. 24, 2012, which is incorporated herein by reference.

### TECHNICAL FIELD OF THE INVENTION

The invention relates to an excrement collector, which relates to the field of medical appliance.

### BACKGROUND OF THE INVENTION

The examination of excrement is significant to the early screening of many diseases. For example, gastrointestinal bleeding can be screened out for a patient by examining the hemoglobin in the excrement. Therefore, the examination of excrement has become a conventional disease examination project for a lot of medical units. Moreover, in order to examine the excrement precisely, it is necessary to develop an excrement collector, which is convenient to collect the excrement and can avoid the leakage of the collected excrement samples during the transportation process.

An excrement collector is disclosed in the patent U.S. Pat. No. 5,514,341, which comprises a collecting cavity, a top cover with a collecting bar, a diaphragm which is configured to divide the cavity of the collector into a sample cavity and a cavity for extra excrement, a filtering device at the bottom of the collecting cavity, and a dripper at the bottom of the collecting cavity positioned outside the filtering device. However, in the preparation process of the excrement collector, it is necessary to integrate the diaphragm with the collecting cavity into a whole and in order to prevent the liquid in the sample cavity from flowing into the cavity for extra excrement, it is necessary to provide a sealing element between the diaphragm and the collecting bar. An excrement collector is disclosed in the patent US20060210448, wherein its excrement collecting part is a plastic bar which can obtain only a few samples, and its sample outlet is hand-separative, which is difficult to separate and may cause the excrement dissolved in liquid in a bottle to splash easily during the separation process, thereby bringing much inconveniences to an operator. Moreover, the examination accuracy may be affected due to the loss of excrement during the splashing process.

Therefore, it is necessary to develop an excrement collector, which is convenient to collect the excrement, simple to manufacture, and is applicable to both a machine examination and a manual examination for excrement.

### SUMMARY OF THE INVENTION

The objective of the invention is to provide an excrement collector, which is simple in structure, easy to manufacture and applicable to both a machine examination and a manual examination for excrement.

The invention provides an excrement collector, which comprises: an upper cover, a collecting tube, a bottom cover and a filtering tube, wherein the filtering tube is a funnel-shaped hollow tube and is fixed on the upper side inside the tube cavity of the collecting tube; a sampling end is formed on the lower part of the upper cover and passes through a through hole at the bottom of the filtering tube; the upper cover is detachably integrated with the upper part of the collecting tube; the bottom cover is detachably integrated with the lower part of the collecting tube; and liquid is provided in the cavity between the collecting tube and the filtering tube.

In one aspect of the invention, the filtering tube comprises a filtering tube body, an opening on the upper part of the filtering tube body, a through hole at the bottom of the filtering tube body, and a flange extending outwards from the top of the filtering tube body, wherein a pallet integrated with the flange is provided on the upper inner wall of a tube cavity of the collecting tube; and the filtering tube and the collecting tube are integrated together by the pallet and the flange.

Preferably, the flange is bonded with the pallet.

In one aspect of the invention, the filtering tube is integrative.

In one aspect of the invention, the filtering tube body of the filtering tube comprises a main tube body and a joint face between the main tube body and the through hole. In addition, the main tube body is cylindrical, and the outer diameter of the main tube body is larger than the inner diameter of the collecting tube.

Preferably, the length of the main tube body is 1/2 to 4/5 of the length of the filtering tube body of the filtering tube.

Preferably, the filtering tube divides the collecting tube into two cavities, wherein the cavity between the collecting tube and the filtering tube is a sample cavity, and the cavity where the collecting tube is superposed with the filtering tube is an observation cavity.

More preferably, the length of the sample cavity is 1/2 to 3/4 of the length of the collecting tube.

In one aspect of the invention, the upper cover comprises a handle end, a sampling end and a sampling rod for connecting the handle end and the sampling end, wherein the length of the sampling rod is longer than the total length of the filtering tube.

Preferably, the sampling end comprises a threaded part extending towards the handle; and when the upper cover is integrated with the upper part of the collecting tube, the sampling end is placed in the liquid between the collecting tube and the filtering tube.

More preferably, the outer diameter of the threaded part is equal to the diameter of the through hole.

In one aspect of the invention, the through hole is a hollow cylinder protruding away from the flange; and the diameter of the through hole is smaller than the diameter of the sampling rod.

In one aspect of the invention, the collecting tube comprises an inlet end, an outlet end and a tube body between the inlet end and the outlet end.

More preferably, both the inlet end and the tube body are cylindrical.

More preferably, the diameter of the inlet end is larger than the diameter of the tube body; and the pallet is a joint face between the inlet end and the tube body.

Most preferably, the diameter of the outlet end is smaller than the diameter of the tube body.

In one aspect of the invention, the inlet end is in threaded connection with the handle end.

Preferably, the inlet end comprises an internal thread; and the handle end comprises an external thread corresponding to the internal thread.

In one aspect of the invention, the outlet end is in threaded connection with the bottom cover.

Preferably, the outlet end comprises an external thread; and the bottom cover comprises an internal thread corresponding to the external thread.

More preferably, an opening communicated with the outside is formed at the bottom of the outlet end and is sealed by a sealing membrane.

In one aspect of the invention, the bottom cover comprises a cylinder wall and a top wall; and the top wall is located on the cross section of the cylinder wall.

Preferably, a spine component for puncturing the bottom of the collecting tube is provided in the center of the outer surface of the top wall.

More preferably, the length of the spine component is less than the height of the cylinder wall around the periphery of the spine component. Most preferably, the inner diameter of the cylinder wall around the spine component is larger than the outer diameter of the outlet end; and the maximum diameter of the spine component is 1/5 to 1/2 of the inner diameter of the outlet end.

In one aspect of the invention, the collecting tube and the filtering tube are made of a transparent material.

Preferably, the collecting tube is mainly made of polyethylene or polypropylene; and the filtering tube is mainly made of polyethylene, or polypropylene, or silica gel.

The invention may further provide a method for using the excrement collector of the invention, including the following steps:

a. removing the bottom cover on the lower part of the collecting tube; and

b. puncturing the bottom part of the collecting tube so as to drop the liquid in the collecting tube to an examination casse for examination.

Preferably, in the step b, the bottom part of the collecting tube is punctured by the spine component on the casse or the spine component on an examination machine.

### BRIEF DESCRIPTION OF THE DRAWINGS

The technical solution of the invention is explained below in combination with the drawings in detail, wherein:

FIG. 1 is an exploded view of a first embodiment of the invention;

FIG. 2 is a sectional view of a filtering tube in the first embodiment of the invention;

FIG. 3 is a sectional view showing that a collecting tube is integrated with a filtering tube in a first embodiment of the invention;

FIG. 4 is a three-dimensional sectional view of a bottom cover in a first embodiment of the invention;

FIG. 5 is a sectional view of an assembling drawing of a first embodiment of the invention;

FIG. 6 is a three-dimensional view of a casse with a spine component of the invention;

FIG. 7 is a sectional view showing that a casse in the FIG. 6 of the invention matches with an excrement collector;

FIG. 8 is an exploded view of a second embodiment of the invention; and

FIG. 9 is a sectional view of a collecting tube in a second embodiment of the invention.

### DETAILED DESCRIPTION OF THE PREFERRED EMBODIMENTS

The invention will be further described below in combination with the embodiments; and the embodiments here are only intended to describe the invention instead of limiting the scope of the invention.

### Embodiment 1

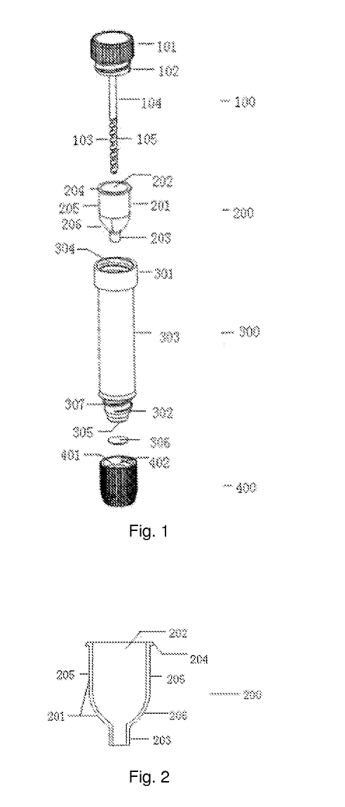

FIG. 1 shows an exploded view of an excrement collector of a first embodiment of the invention. As shown, the excrement collector comprises a collecting tube 300, an upper cover 100 detachably integrated on the upper part of the collecting tube, a filtering tube 200 fixed on the upper side inside the collecting tube and a bottom cover 400 detachably integrated on the lower part of the collecting tube.

The filtering tube 200 is a funnel-shaped hollow tube, and the filtering tube comprises a filtering tube body 201, an opening 202 on the upper part of the filtering tube body, a through hole 203 at the bottom of the filtering tube body and a flange 204 extending outwards from the top of the filtering tube. The filtering tube body of the filtering tube comprises a main tube body 205 and a joint face 206 between the main tube body 205 and the through hole 203.

The upper part of the upper cover 100 is a handle end 101, a thread 102 is provided at the lower side of the handle end for screwing with the collecting tube 300; a slender sampling end 103 is provided at the lower part of the upper cover 100, and a sampling rod 104 is integrated with the handle end and the sampling end; and the sampling end 103 comprises a threaded part 105 extending from the bottom of the sampling end to the handle end. The length of the sampling rod is longer than the total length of the filtering tube.

The collecting tube 300 comprises an inlet end 301, an outlet end 302, a tube body 303 for connecting with the inlet end and the outlet end, an internal thread 304 at the inlet end, an opening 305 at the outlet end, a sealing membrane 306 sealing the opening and a thread 307 at the outlet end. The diameter of the inlet end is larger than the diameter of the tube body, while the diameter of the outlet end is smaller than the diameter of the tube body.

The bottom cover 400 comprises an opening 402 for accommodating the outlet end 302 of the collecting tube and a thread 401 inside the opening.

As shown in FIG. 2, the filtering tube 200 is a hollow tube. The opening 202 on the upper part of the filtering tube body is communicated with the through hole 203 at the bottom of the tube. The filtering tube body 201 of the filtering tube comprises a main tube body 205 and a joint face 206 between the main tube body and the through hole 203. Both the main tube body and the through hole 203 are cylindrical.

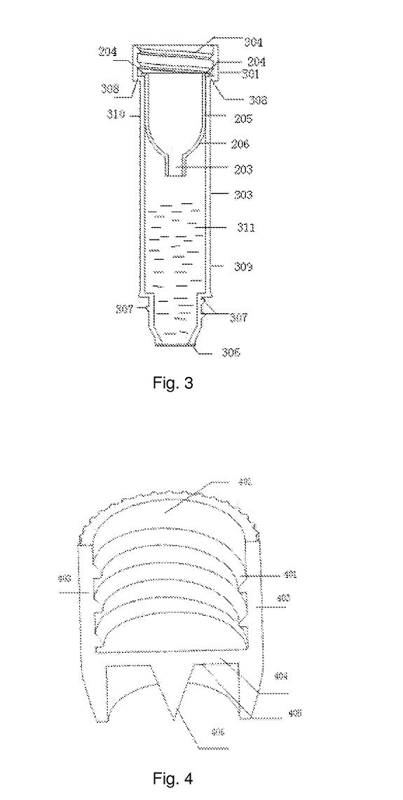

As shown in FIG. 3, the filtering tube 200 is a funnel-shaped hollow tube. A flange 204 extends outwards from the top of the filtering tube. A pallet 308 integrated with the flange is provided on the upper inner wall of the cavity of the collecting tube and the pallet 308 is a joint face between the inlet end 301 and the tube body 303; and the filtering tube and the collecting tube are integrated together by the flange and the pallet. The main tube body 205 of the filtering tube is cylindrical, and the outer diameter of the main tube body 205 of the filtering tube is larger than the inner diameter of the collecting tube. The contact surface between the filtering tube and the collecting tube is the whole outer surface of the main tube body 205. The main tube body is integrated with the collecting tube by interference fit. The sealing membrane 306 is sealed at the opening 305 of the outlet end. The filtering tube divides the collecting tube into two cavities, wherein the cavity between the collecting tube and the filtering tube is a sample cavity 309, and the cavity where the collecting tube is superposed with the filtering tube is an observation cavity 310. Liquid 311 is provided in the sample cavity 309.

As shown in FIG. 4, the bottom cover 400 comprises a cylinder wall 403 and a top wall 404 on the cross section of the cylinder wall. The external surface 405 of the top wall 404 further comprises a spine component 406, the length of the spine component is less than or equal to the height of the cylinder wall around the periphery of the spine component. The maximum diameter of the spine component is 1/3 of the inner diameter of the outlet end. The inner diameter of the cylinder wall around the spine component is larger than the outer diameter of the outlet end. Therefore, when the bottom cover is unscrewed from the outlet end to be turned over, the outlet end can extend into the cavity where the spine component is located, and the spine component punctures the sealing membrane so that the liquid in the collecting tube flows to examination equipment to be examined.

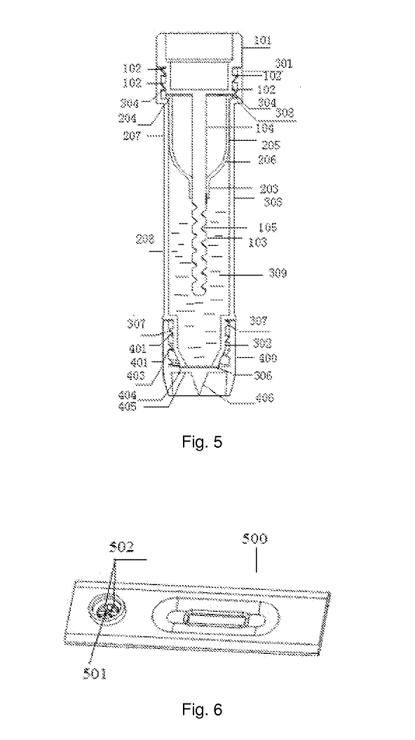

As shown in FIG. 5, the inlet end 301 of the collecting tube 300 comprises a thread 304, which corresponds to the thread 102 on the handle of the upper cover. When the sampling end 103 passes through the through hole 203 at the bottom of the filtering tube, the thread 102 of the upper cover is integrated with the thread 304 at the inlet end of the collecting tube, and the sampling rod 104 is integrated with the inner surface of the through hole 203. Because the inner diameter of the through hole is smaller than the outer diameter of the sampling rod, the sampling rod is in interference fit with the through hole.

The filtering tube is integrated with the collecting tube by the flange 204 of the filtering tube and the pallet 308 of the collecting tube. At this point, the flange 204 can play a role of a sealing ring between the inlet end and the handle.

The outer diameter of the main tube body 205 of the filtering tube is larger than the inner diameter of the tube body 303 of the collecting tube, so the filtering tube is in interference fit with the collecting tube. Thereby, the liquid 311 can be restricted within the sample cavity 309, the liquid 311 cannot leak from the joint face between the main tube body 205 and the collecting tube 303 and the liquid 311 cannot leak to the observation cavity 310 from the joint face between the sampling rod and the through hole.

The outer diameter of threaded part 105 of the sampling end 103 of the upper cover 100 is equal to the diameter of the through hole. Since the excrement collected by the sampling end 103 is gathered in the threaded part 105, when the sampling end 103 is inserted into the through hole 203 of the filtering tube 200, the excrement gathered on the outer diameter of the thread is intercepted to the joint face 206 of the filtering tube 200, and the excrement between the outer diameter and the inner diameter of the thread enters the sample cavity 309 through the through hole 203 to be dissolved in the liquid 311. Consequently, the excrement dissolved in the liquid 311 can be examined quantitatively.

The outlet end of the collecting tube comprises an external thread 307, and the bottom cover comprises an internal thread 401 corresponding to the external thread, and the bottom cover is in threaded connection with the outlet end of the collecting tube.

In case of sampling, the upper cover 100 is unscrewed, and the handle 101 is held to insert the sampling end 103 of the upper cover 100 into an excrement sample, so that the threaded part 105 of the sampling end is filled with excrement samples; and then, the sampling end 105 filled with the samples is inserted to the through hole 203 of the filtering tube 200. Because the outer diameter of the thread is equal to the inner diameter of the through hole, the extra excrement samples are reserved on the joint face 206 of the filtering tube, and the characters of the extra excrement can be observed through the transparent observation cavity. When the liquid volume in the sample cavity 309 is a fixed value, the substance to be examined in the excrement can be studied quantitatively because the quantity of excrement in the thread is also a fixed value.

The sampling end 103 is inserted into the sample cavity 309 completely, and the upper cover 100 is screwed tightly to shake the collector so that the excrement between the outer diameter and the inner diameter of the thread is dissolved in the liquid 311 in the sample cavity 309. Finally, the bottom cover 400 is unscrewed and turned over to reveal the spine component 406, the outlet end 302 of the collecting tube is inserted into the cavity enclosed by the spine component, the cylinder wall 403 and the top wall 404, and the sealing membrane 306 is punctured by the spine component, and the collecting tube is placed vertically downward so that the liquid in the sample cavity flows out from the punctured opening to be examined.

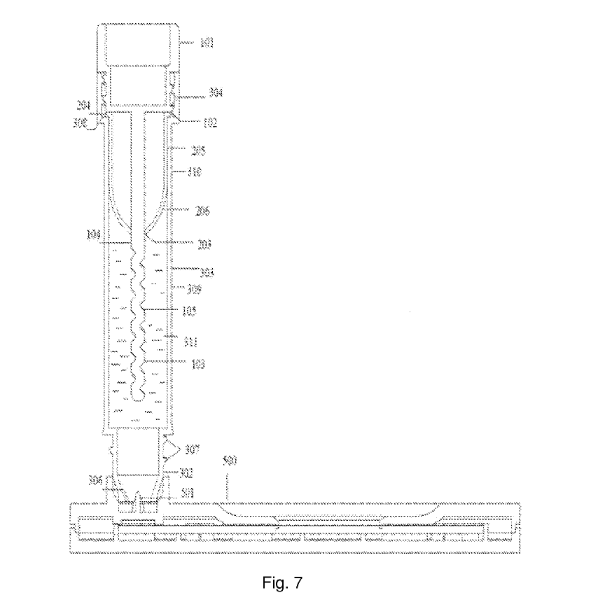

The excrement collector of the invention is also applicable to a machine examination. In the examination process of the machine, firstly, the bottom cover 400 of the excrement collector is unscrewed, wherein the excrement is dissolved in the liquid 311 of excrement collector; secondly, the excrement collector is placed on the rack of the examination machine; and finally, an examination casse 500 with a spine component is placed on the examination grillage of the examination machine, wherein the casse is as shown in FIG. 6. The excrement collector on the rack gets close to the casse 500, and the spine component 501 of the casse punctures the sealing membrane 306 of the excrement collector, as shown in FIG. 7, consequently, the liquid in the sample cavity directly drips from the punctured opening to a dripping hole 502 on the casse to be examined.

In the examination process of the machine, the excrement collector can be placed on the rack of the examination machine, and the sealing membrane 306 of the excrement collector is punctured by a spine component on the machine, so that the liquid in the sample cavity directly drips from the punctured opening to the dripping hole 502 of the casse to be examined.

### Embodiment 2

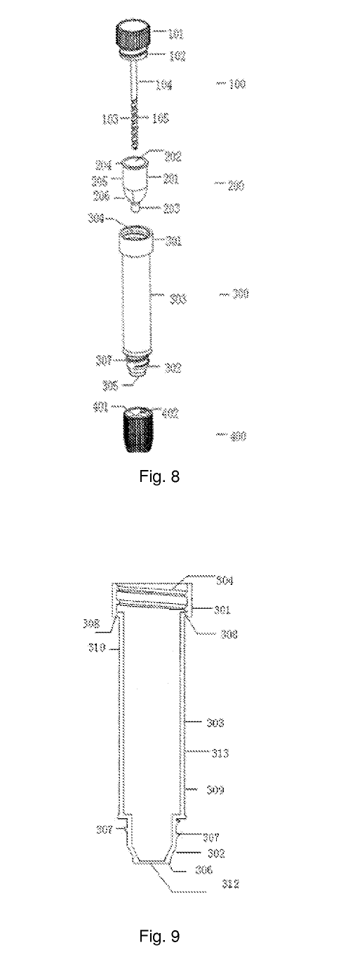

As shown in FIG. 8, this embodiment is substantively the same as the first embodiment, and the difference lies in that the outlet end 302 of the collecting tube here does not comprise an opening 305 nor comprise a sealing membrane 306.

With reference to FIG. 9, in this embodiment, the outlet end 302 of the collecting tube is closed and is integrated with the collecting tube into a whole; and the thickness of the tube wall 312 of the outlet end is thinner than the thickness of the tube wall 313 of the collecting tube. Therefore, the spine component 406 of the bottom cover 400 can puncture the tube wall 312 of the outlet end.

To sum up, as compared with the prior arts, the excrement collector provided in the invention has the following advantages that:

1. The excrement collector has a sealed structure, which can avoid leakage and the overflow of odor and the excrement collector is safe and sanitary;

2. The excrement collector is simple in structure so as to be convenient to carry and suitable for collecting the sample at different places;

3. The excrement collector is used for quantitatively examining the substance to be examined in the excrement;

4. The excrement collector is applicable to both a machine examination and a person examination; and

5. The excrement sample can be observed from the observation cavity by eyes, so that a primary diagnosis can be made according to the color, shape and the like of the excrement sample.

Although the exemplary embodiments of the invention have been described with reference to the drawings, those skilled in the art shall understand that some changes may also be made to the forms and details of the embodiments within the spirit and scope of the invention.

1. An excrement collector, comprising: an upper cover, a collecting tube, a bottom cover and a filtering tube, wherein the filtering tube is a funnel-shaped hollow tube and is fixed on the upper side inside the tube cavity of the collecting tube; a sampling end is formed on the lower part of the upper cover and passes through a through hole at the bottom of the filtering tube; the upper cover is detachably integrated with the upper part of the collecting tube; the bottom cover is detachably integrated with the lower part of the collecting tube; and liquid is provided in the cavity between the collecting tube and the filtering tube.

2. The excrement collector according to claim 1, wherein the filtering tube comprises a filtering tube body, an opening on the upper part of the filtering tube body, a through hole at the bottom of the filtering tube body, and a flange extending outwards from the top of the filtering tube body; a pallet integrated with the flange is provided on the upper inner wall of the tube cavity of the collecting tube; the filtering tube and the collecting tube are integrated together by the flange and the pallet; preferably, the flange is bonded with the pallet; and more preferably, the filtering tube is integrative.

3. The excrement collector according to claim 1 or 2, wherein the filtering tube body of the filtering tube comprises a main tube body and a joint face between the main tube body and the through hole; the main tube body is cylindrical, and the outer diameter of the main tube body is larger than the inner diameter of the collecting tube; preferably, the length of the main tube body is 1/2 to 4/5 of the length of the filtering tube body of the filtering tube; more preferably, the filtering tube divides the collecting tube into two cavities, the cavity between the collecting tube and the filtering tube is a sample cavity, and the cavity where the collecting tube is superposed with the filtering tube is an observation cavity; and most preferably, the length of the sample cavity is 1/2 to 3/4 of the length of the collecting tube.

4. The excrement collector according to claim 1, wherein the upper cover comprises a handle end, a sampling end and a sampling rod for connecting the handle end and the sampling end, and the length of the sampling rod is longer than the total length of the filtering tube; preferably, the sampling end comprises a threaded part extending towards the handle end, and when the upper cover is integrated with the upper part of the collecting tube, the sampling end is placed in the liquid between the collecting tube and the filtering tube; and more preferably, the outer diameter of the threaded part is equal to the diameter of the through hole.

5. The excrement collector according to claim 4, wherein the through hole is a hollow cylinder protruding away from the flange, and the diameter of the through hole is smaller than the diameter of the sampling rod.

6. The excrement collector according to claim 1, wherein the collecting tube comprises an inlet end, an outlet end and a tube body between the inlet end and the outlet end; preferably, both the inlet end and the tube body are cylindrical, the diameter of the inlet end is larger than the diameter of the tube body, and the pallet is a joint face between the inlet end and the tube body; and most preferably, the diameter of the outlet end is smaller than the diameter of the tube body.

7. The excrement collector according to claim 6, wherein the inlet end is in threaded connection with the handle end; and preferably, the inlet end comprises an internal thread, and the handle end comprises an external thread corresponding to the internal thread.

8. The excrement collector according to claim 6 or 7, wherein the outlet end is in threaded connection with the bottom cover; preferably, the outlet end comprises an external thread, and the bottom cover comprises an internal thread corresponding to the external thread; and more preferably, an opening communicated with the outside is formed at the bottom of the outlet end and is sealed by a sealing membrane.

9. The excrement collector according to claim 1, wherein the bottom cover comprises a cylinder wall and a top wall, and the top wall is located on the cross section of the cylinder wall; preferably, a spine component for puncturing the bottom of the collecting tube is provided in the center of the outer surface of the top wall; more preferably, the length of the spine component is less than the height of the cylinder wall around the periphery of the spine component; and most preferably, the inner diameter of the cylinder wall around the spine component is larger than the outer diameter of the outlet end, and the maximum diameter of the spine component is 1/5 to 1/2 of the inner diameter of the outlet end.

10. The excrement collector according to claim 1, wherein the collecting tube and the filtering tube are made of a transparent material; and preferably, the collecting tube is mainly made of polyethylene or polypropylene, and the filtering tube is mainly made of polyethylene, or polypropylene, or silica gel.

11. A method for using the excrement collector according to claim 1, comprising the following steps:
a. removing the bottom cover on the lower part of the collecting tube, and
b. puncturing the bottom part of the collecting tube to drop the liquid in the collecting tube to an examination casse for examination; and preferably, in the step b, the bottom part of the collecting tube is punctured by the spine component on the casse or the spine component on an examination machine.
